(19) United States
(12) Patent Application Publication
(10) Pub. No.: US 2019/0388387 A1
(43) Pub. Date: Dec. 26, 2019

(54) COMPOSITION FOR INHIBITING MYOFIBROSIS

(71) Applicant: SUNTORY HOLDINGS LIMITED, Osaka (JP)

(72) Inventors: Daisuke YOSHIDA, Kyoto (JP); Yuta OTSUKA, Kyoto (JP)

(73) Assignee: SUNTORY HOLDINGS LIMITED, Osaka (JP)

(21) Appl. No.: 16/492,284

(22) PCT Filed: Mar. 8, 2018
(86) PCT No.: PCT/JP2018/008960

§ 371 Date: Sep. 9, 2019

(30) Foreign Application Priority Data
Mar. 10, 2017 (JP) 2017-046265

(51) Int. Cl. A61K 31/353; A61K 9/00

(52) U.S. Cl. CPC A61K 31/353; A61K 9/0095; A61K 9/0056; A61K 9/0053

(57) ABSTRACT

The present invention aims to provide a composition for inhibiting myofibrosis, which contains, as an active ingredient, a component that is safely ingestible for a long period of time; and a method of safely inhibiting myofibrosis. The present invention provides a composition for inhibiting myofibrosis containing quercetin or a glycoside thereof as an active ingredient.

### TECHNICAL FIELD

The present invention relates to a composition for inhibiting myofibrosis. The present invention also relates to a method of inhibiting myofibrosis and the like.

### BACKGROUND ART

The super-aged society of Japan with advanced medical technology and an increasing proportion of the elderly aged 75 or over faces challenge to extend healthy life span and to improve the quality of life (QOL). One of the factors that shorten the healthy life span is a condition where an elderly is at high risk of requiring nursing care due to locomotor disorder, which is called “locomotive syndrome”. A patient of locomotive syndrome suffers from age-related phenomena, such as pain from motor organ disease and reduced motor function such as reduced muscle strength, reduced stamina, or reduced motion speed, and the patient may fall into a negative chain as these phenomena are combined together, with the result that the patient cannot perform daily activities and is likely to end up in requiring nursing care. Thus, prevention of locomotive syndrome is important. Current measures to prevent impairment of motor function include rehabilitation such as mild exercise and electrostimulation. Further, studies based on nutritional point of view have been made and found components that may be able to prevent muscle atrophy and a reduction in muscle strength (Patent Literatures 1 and 2).

The level of motor function is known to be not only simply proportional to the muscle mass but also associated with muscle quality. Thus, improvement of muscle quality, in addition to prevention of muscle atrophy, is considered to be important for improving motor function. The muscle quality means muscle strength per muscle cross-sectional area or muscle mass, or tension per muscle fiber, and is known to decrease with aging (Non-Patent Literature 1). A factor of an age-related reduction in muscle quality is considered to be accumulation of fibrous components or lipid droplets in muscle.

In muscle, muscle satellite cells, which are stem cells, exist between basal lamina and cell membrane of muscle fibers. The muscle satellite cells are activated by stimuli such as muscle injury and proliferate. At the same time, these cells differentiate into myoblasts and myotubes to form new muscle fibers. Promoting differentiation of muscle satellite cells into myoblasts and myotubes is considered to be important for thickening or regenerating muscle which is important to achieve prevention of muscle atrophy or improvement of motor function. Components that promote differentiation of muscle satellite cells into myoblasts and myotubes have been reported. Examples include perennial sword-leaf dogbane of the family Apocynaceae or its extract and an extract of black tea produced through fermentation from tea leaves obtainable from hybrid plants of the genus Camellia (Patent Literatures 3 and 4).

Muscle satellite cells proliferate through self-replication when activated, and differentiate into myoblasts and myotubes. At the same time, muscle satellite cells can also differentiate into adipose cells or myofibroblasts, according to a report (Non-Patent Literature 2). The cells that have differentiated into adipose cells produce lipid droplets, and the cells that have differentiated into myofibroblasts produce fibrous components such as collagen. In this manner, lipid droplets accumulate in muscle (adiposis of muscle), or extracellular matrix such as fibrous components accumulate excessively in muscle (fibrosis of muscle (also referred to as “myofibrosis”)), reducing muscle quality. It has been reported that along with aging, muscle satellite cells start differentiating not into myoblasts but into cells that produce fibrous components, with the result that muscle fibrosis is promoted (Non-Patent Literature 3). Advanced studies have been made on tissue fibrosis particularly in the liver and the heart. Yet, studies on muscle fibrosis have not been much advanced. No studies have been made on myofibrosis with a focus on differentiation of muscle satellite cells into myofibroblasts, and no components have been found that inhibit differentiation of muscle satellite cells into myofibroblasts.

Quercetin is a type of flavonoid and is present as-is or as a glycoside in many plants such as onions. Quercetin has been reported to have physiological properties such antioxidant, anti-inflammatory, anti-tumor, and vasodilator effects. Meanwhile, an effect of quercetin on differentiation of muscle satellite cells into myofibroblasts has remained unknown.

### CITATION LIST

### Patent Literatures

Patent Literature 1: JP 2002-338464 A
Patent Literature 2: WO 2005/074962
Patent Literature 3: JP 2014-15428 A
Patent Literature 4: JP 2013-91608 A

### Non-Patent Literatures

Non-Patent Literature 1: Journal of Applied Physiology, 1999, 86(1), pp. 188-194
Non-Patent Literature 2: Journal of cell science, 2011, 124, pp. 3654-3664
Non-Patent Literature 3: Journal of Science, 2007, 317, 5839, pp. 807-810

### SUMMARY OF INVENTION

### Technical Problem

The present invention aims to provide a composition for inhibiting myofibrosis, which contains, as an active ingredient, a component that is safely ingestible for a long period of time. The present invention also aims to provide a method of safely inhibiting myofibrosis.

### Solution to Problem

The present inventors used rat muscle-derived satellite cells and found that quercetin has an effect of inhibiting the differentiation process from muscle satellite cells into myofibroblasts and is useful in inhibiting myofibrosis. The present invention was thus completed.

Specifically, the present invention includes, but not limited to, a composition for inhibiting myofibrosis, a method of inhibiting myofibrosis, and the like defined below:

(1) a composition for inhibiting myofibrosis, containing quercetin or a glycoside thereof as an active ingredient;
(2) the composition for inhibiting myofibrosis according to (1), wherein the composition inhibits the differentiation process from muscle satellite cells into myofibroblasts;
(3) the composition for inhibiting myofibrosis according to (1) or (2), wherein the composition has an effect of improving muscle quality;
(4) the composition for inhibiting myofibrosis according to any one of (1) to (3), wherein the composition has an effect of improving motor function;
(5) the composition for inhibiting myofibrosis according to any one of (1) to (4), wherein the composition has an effect of increasing muscle mass;
(6) the composition for inhibiting myofibrosis according to any one of (1) to (5), wherein the composition has an effect of inhibiting muscle atrophy;
(7) the composition for inhibiting myofibrosis according to any one of (1) to (6), wherein the composition has an effect of inhibiting accumulation of extracellular matrix in muscle;
(8) the composition for inhibiting myofibrosis according to (7), wherein the extracellular matrix is at least one of collagen or an advanced glycation end product;
(9) the composition for inhibiting myofibrosis for any one of (1) to (8), which is a food, beverage, or oral pharmaceutical product;
(10) the composition for inhibiting myofibrosis according to any one of (1) to (9), wherein the composition is labeled as having one or more of the following effects: inhibition, prevention, or amelioration of myofibrosis; improvement of muscle quality; improvement of motor function; increase in muscle mass; and inhibition of muscle atrophy, or is labeled for use in achieving one or more the above effects;
(11) use of quercetin or a glycoside thereof for inhibiting myofibrosis;
(12) use of quercetin or a glycoside thereof for inhibiting the differentiation process from muscle satellite cells into myofibroblasts;
(13) a method of inhibiting myofibrosis, including: administering or feeding quercetin or a glycoside thereof; and
(14) a method of inhibiting the differentiation process from muscle satellite cells into myofibroblasts, including: administering or feeding quercetin or a glycoside thereof.

### Advantageous Effects of Invention

The present invention provides a composition for inhibiting myofibrosis, which contains quercetin or a glycoside thereof as an active ingredient. Quercetin and glycosides thereof have an effect of inhibiting the differentiation process from muscle satellite cells into myofibroblasts, are effective in inhibiting myofibrosis, and are highly safe. Thus, the present invention can provide a composition for inhibiting myofibrosis, which contains, as an active ingredient, a component that is safely ingestible for a long period of time. The present invention can safely inhibit myofibrosis.

### BRIEF DESCRIPTION OF DRAWINGS

FIG. 1 shows micrographs of muscle satellite cells stained with Masson's Trichrome after being cultured for 24 hours in a differentiation-inducing medium with addition of a TGF β (TGF-β) signal inhibitor or quercetin ((a): TGF β-free group; (b): TGF β-added group; (c): SB525334-added group, (d): quercetin 50 μM-added group; (e): quercetin 100 μM-added group).

FIG. 2 shows graphs showing analysis results of the expression level of an intracellular fibrosis marker gene after muscle satellite cells were cultured for 24 hours in a differentiation-inducing medium with addition of a TGF β signal inhibitor or quercetin ((a): Acta2 gene; (b) Colla1 gene; **: P<0.01, vs. TGF ((+)).

### DESCRIPTION OF EMBODIMENTS

The composition for inhibiting myofibrosis of the present invention contains quercetin or a glycoside thereof as an active ingredient.

Quercetin and glycosides thereof have an effect of inhibiting the differentiation process from muscle satellite cells into myofibroblasts and exhibit an effect of inhibiting myofibrosis.

The composition for inhibiting myofibrosis of the present invention is used to inhibit accumulation of extracellular matrix including fibrous components such as collagen in muscle (i.e., myofibrosis). The composition for inhibiting myofibrosis of the present invention is suitably used to inhibit myofibrosis by inhibiting the differentiation process from muscle satellite cells into myofibroblasts.

The degree of inhibition of the differentiation process from muscle satellite cells into myofibroblasts can be evaluated based on myofibroblast abundance. The myofibroblast abundance can be evaluated, for example, by measuring the expression level of a marker gene specific to myofibroblasts, by measuring the protein content encoded by the gene, or by observing the morphology of muscle satellite cells. In examples described later, in order to quantitatively evaluate the myofibroblast abundance, the expression level of Acta2 gene which is a marker specific to myofibroblasts and the expression level of Colla1 gene (type I collagen gene) involved in the production of type I collagen were measured for evaluation.

The Acta2 gene encodes for α-smooth muscle actin (α-SMA) protein, and α-SMA is a protein that is expressed in fibroblasts or myofibroblasts. Accumulation of activated fibroblasts or myofibroblasts in a fibrosis site results in mass production of type I collagen, and this is considered to be the cause of tissue fibrosis. Thus, inhibition of the differentiation process from muscle satellite cells into myofibroblasts includes inhibition of expression of Acta2 gene and Colla1 gene in muscle satellite cells.

Transforming growth factor β (TGF-β) stimulation induces differentiation of muscle satellite cells in mammals into myofibroblasts. Quercetin and glycosides thereof have the effect of inhibiting the differentiation process from muscle satellite cells into myofibroblasts which is induced by TGF-β (also described as TGF β).

As used herein, “muscle satellite cells” means mesenchymal stem cells that exist between basal lamina and cell membrane of muscle fibers. Muscle satellite cells are known to be capable of differentiating not only into myoblasts but also into adipose cells, bone cells, myofibroblasts, and the like. For example, muscle satellite cells differentiate into myofibroblasts when cultured in a myofibroblast-inducing medium. Muscle satellite cells differentiate into adipocyte-like cells when cultured in an adipose differentiation-inducing medium. Differentiation of muscle satellite cells into myofibroblasts or adipocyte-like cells causes a reduction in muscle quality, a reduction in muscle mass, or muscle atrophy, resulting in a reduction in motor function.

Myofibrosis can be inhibited by inhibiting the differentiation process from muscle satellite cells into myofibroblasts. Additionally, inhibiting the differentiation process from muscle satellite cells into myofibroblasts allows muscle satellite cells to maintain their stem cell capacity and/or to be preferentially induced to differentiate into myoblasts. Thus, a reduction in muscle quality, reduction in muscle mass, and muscle atrophy can be inhibited by inhibiting the differentiation process from muscle satellite cells into myofibroblasts. Achieving these effects result in achieving an effect of improving motor function.

As used herein, the “myofibrosis induction” means TGF-β-stimulated induction of differentiation of muscle satellite cells into myofibroblasts. Presumably, fibrosis of many tissues is caused by activation of fibroblasts or myofibroblasts by increased TGF-β signaling. It has been reported that in organs such as lung and kidney, overexpression of TGF-β causes fibrosis, whereas inhibition of TGF-β signaling inhibits fibrosis.

As used herein, the “muscle quality” means muscle strength per muscle cross-sectional area or muscle mass, or tension per muscle fiber. Thus, as used herein, the “improvement of muscle quality” means an increase in muscle strength per muscle cross-sectional area or muscle mass or an increase in tension per muscle fiber. Improvement of muscle quality can also mean that the increase rate of muscle strength is higher than the increase rate of muscle cross-sectional area or muscle mass.

As used herein, the “motor function” means function to perform daily activities such as walking, climbing up and down the stairs, and standing up. For example, the function can be measured from the knee extension muscle strength, grip strength, walking speed, or the like. Thus, as used herein, the “improvement of motor function” means improved results of these measurement items.

As used herein, the “increase in muscle mass” means an increase in the number of muscle fibers per unit area, increase in muscle fiber cross-sectional area, increase in muscle cross-sectional area, or increase in muscle weight. An increase in muscle mass occurs due to an increase in the protein content in muscle tissue which occurs when the rate of muscle protein synthesis is higher than the rate of muscle protein degradation. Presumably, inhibiting the differentiation process from muscle satellite cells into myofibroblasts substantially increases the percentage of muscle satellite cells that differentiate into myoblasts, leading to an increase in muscle mass. This is effective in restoring reduced muscle mass.

As used herein, the “muscle atrophy” means a decrease in the protein content in muscle tissue which occurs when the rate of muscle protein synthesis is lower than the rate of degradation. Thus, the “inhibition of muscle atrophy” means to normalize the balance between synthesis and degradation so as to inhibit a decrease in the protein content in muscle tissue by increasing the rate of muscle protein synthesis and/or decreasing the rate of muscle protein degradation.

As used herein, the “extracellular matrix accumulation” means an excessive accumulation of extracellular matrix including collagen and advanced glycation end products (AGEs) in muscle. In particular, collagen and advanced glycation end products are known to accumulate along with aging.

As used herein, the “collagen” means a molecule having 3- or 4-hydroxyproline and 5-hydroxylysine residues as amino acid residues. Usually, 3- or 4-hydroxyproline and 5-hydroxylysine residues are hardly contained in other proteins. Generally, the collagen content in animal tissue is presumably deducible by measuring the amount of 4-hydroxyproline residues.

As used herein, the “advanced glycation end product” means a substance produced by non-enzymatic reaction of a reducing sugar such as glucose with an amino group of a protein. In particular, collagen is glycosylated, resulting in advanced glycation end products which are known to easily accumulate. It has been reported that accumulation of advanced glycation end products in muscle is involved in reduced muscle function (Journal of applied physiology, 2007, vol. 103 (6): 2068-76).

As used herein, the “quercetin” is also referred to as vitamin P. It is quercetin as a compound belonging to the flavonol group of polyphenols. Quercetin is a compound represented by the following formula (I).

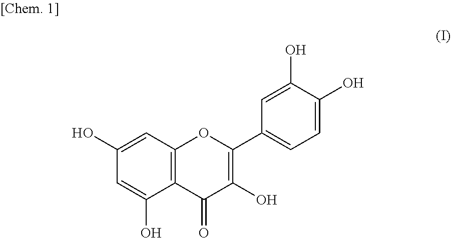 [SMILES: O=c1c(O)c(-c2ccc(O)c(O)c2)oc2cc(O)cc(O)c12]

In the present invention, the “quercetin glycoside” means a glycoside of the quercetin. The quercetin glycoside is a compound represented by the following formula (II). In the following formula (II), (X)n represents a sugar chain, and n is an integer of 1 or more.

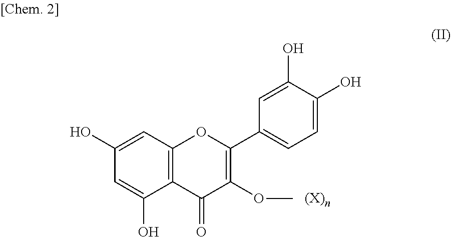 [SMILES: COc1c(-c2ccc(O)c(O)c2)oc2cc(O)cc(O)c2c1=O]

Examples of sugars constituting the sugar chain that is glycosidically linked to quercetin and that is represented by X include glucose, rhamnose, galactose, and glucuronic acid. Glucose and rhamnose are preferred. In addition, n is not limited as long it is 1 or more, and is preferably 1 to 16, more preferably 1 to 8. When n is 2 or more, the X moiety may consist of one sugar or multiple sugars. In other words, when n is 2 or more, (X)n may be a sugar chain consisting of one sugar or a sugar chain consisting of multiple sugars. The quercetin glycoside in the present invention includes a quercetin glycoside obtained by treating an existing quercetin glycoside with an enzyme or the like for transglycosylation. Specific examples of the quercetin glycoside as used herein include rutin, enzyme-treated rutin, quercitrin, and isoquercitrin. In the present invention, the quercetin glycoside is particularly preferably an enzyme-treated product of rutin. Preferred examples of the enzyme-treated product of rutin include: isoquercitrin obtained by treating a quercetin glycoside with an enzyme and thus removing a rhamnose sugar chain moiety; a product obtained by treating isoquercitrin with a glycosyltransferase to bind a sugar chain consisting of one to seven glucose molecules thereto; and a product mainly containing a mixture thereof.

In the present invention, the quercetin or a glycoside thereof may be of one type or two or more types of compounds. In the case of two or more compounds, for example, quercetin and one or more quercetin glycosides may be used, or two or more quercetin glycosides may be used.

The ingested quercetin glycoside, after being absorbed into the body from the digestive tract, is converted into quercetin by the activity of a digestive enzyme or a metabolic enzyme, and exerts the same effect as quercetin in the body.

The origin and the production method of quercetin or a glycoside thereof for use in the present invention are not limited. Examples of plants known to contain a large amount of quercetin or a glycoside thereof include buckwheat, Japanese pagoda trees, caper, apples, tea, onions, grapes, broccoli, nalta jute, raspberries, cowberries, cranberries, prickly pears, leaf vegetables, and citrus fruits. Quercetin or a glycoside thereof can be obtained from these plants.

As shown in the examples described later, quercetin inhibited morphological changes of muscle satellite cells under TGF-β-mediated fibrosis-inducing conditions, and inhibited gene expression of Acta2 gene and Colla1 gene. This specifically means that quercetin has the effect of inhibiting the differentiation process from muscle satellite cells into myofibroblasts. A cause of tissue fibrosis is considered to be excessive production of extracellular matrix such as collagen by myofibroblasts or fibroblasts. Inhibiting the differentiation process from muscle satellite cells into myofibroblasts inhibits an increase in myofibroblasts. This is accompanied by inhibition of accumulation of extracellular matrix accumulation such as collagen and advanced glycation end products by myofibroblasts, resulting in inhibiting myofibrosis. Inhibiting myofibrosis can also prevent or ameliorate symptoms attributable to myofibrosis. Examples of symptoms attributable to myofibrosis include a reduction in muscle quality, a reduction in muscle mass, muscle atrophy, and a reduction in motor function. Prevention encompasses prevention of symptom onset, delay of symptom onset, and decreased incidence of diseases. Amelioration encompasses relief of symptoms, suppression of progression of symptoms, and cure of symptoms.

Presumably, inhibiting the differentiation process of muscle satellite cells into myofibroblasts further allows muscle satellite cells to maintain their stem cell capacity or to be preferentially induced to differentiate into myoblasts, which presumably promotes muscle synthesis in addition to inhibiting accumulation of fibrous components. Thus, effects such as inhibition of myofibrosis, improvement of muscle quality, increase in muscle mass, and inhibition of muscle atrophy can be achieved by inhibiting the differentiation process from muscle satellite cells into myofibroblasts. Achieving these effects results in achieving an effect of improving motor function.

Quercetin or a glycoside thereof is useful in inhibiting the differentiation process from muscle satellite cells into myofibroblasts, and can be used for such a purpose.

Quercetin or a glycoside thereof is useful in inhibiting myofibrosis. Quercetin or a glycoside thereof can be used to inhibit myofibrosis. Quercetin or a glycoside thereof can be used in various applications such as foods, beverages, pharmaceutical products, quasi-pharmaceutical products, feed, and cosmetics which are used to inhibit the differentiation process from muscle satellite cells into myofibroblasts or to inhibit myofibrosis. Quercetin or a glycoside thereof can be suitably used as an active ingredient of these products. Inhibition of myofibrosis is preferably inhibition of skeletal myofibrosis.

As described above, quercetin or a glycoside thereof has the effect of inhibiting the differentiation process from muscle satellite cells into myofibroblasts, and is thus useful in improving muscle quality, improving motor function, increasing muscle mass, inhibiting muscle atrophy, and the like.

In one embodiment, the present invention provides a composition for inhibiting myofibrosis, which contains quercetin or a glycoside thereof as an active ingredient. The composition for inhibiting myofibrosis of the present invention is useful in preventing or ameliorating myofibrosis.

In one embodiment, the present invention provides a composition for inhibiting myofibrosis, which contains quercetin or a glycoside thereof as an active ingredient and which has one or more of the following effects: effect of improving muscle quality; effect of improving motor function; effect of increasing muscle mass; effect of inhibiting muscle atrophy; and effect of inhibiting accumulation of extracellular matrix in muscle.

The composition for inhibiting myofibrosis of the present invention may be provided as an agent, for example, but it is not limited thereto. The agent may be provided as a composition by itself or as a composition containing the agent.

The composition for inhibiting myofibrosis of the present invention can be provided, for example, in the form of a food, beverage, pharmaceutical product, quasi-pharmaceutical product, feed, cosmetic, or any other forms. The composition for inhibiting myofibrosis of the present invention may be a food, beverage, pharmaceutical product, quasi-pharmaceutical product, feed, cosmetic, or the like by itself, or may be an additive such as a formulation or material which is added to these products. In one embodiment, the composition for inhibiting myofibrosis of the present invention is preferably an oral composition.

In one embodiment, the composition for inhibiting myofibrosis of the present invention is preferably a food, beverage, pharmaceutical product (preferably an oral pharmaceutical product), or quasi-pharmaceutical product, more preferably a food, beverage, or oral pharmaceutical product, still more preferably a food or beverage.

As long as the effects of the present invention are not impaired, the composition for inhibiting myofibrosis of the present invention may contain any additives and any components, in addition to the active ingredient, i.e., quercetin or a glycoside thereof. Such additives and components are generally those usable in foods, beverages, pharmaceutical products, quasi-pharmaceutical products, feed, cosmetics, and the like. Examples of such additives or components include physiologically active ingredients such as vitamins (e.g., vitamin E and vitamin C), minerals, and nutrients, and additives that are added during preparation such as excipients, binders, emulsifiers, tonicity agents (isotonizing agent), buffers, solubilizers, preservatives, stabilizers, antioxidants, colorants, coagulants, coating agents, and perfumes. Examples of such components also include proteins such as casein protein, whey protein, and soy protein and peptides thereof; and amino acids including branched chain amino acids such as valine, leucine, and isoleucine and metabolites thereof. These may be used alone or in combination of two or more thereof.

In addition to those mentioned above, components such as material usable in foods, beverages, pharmaceutical products, quasi-pharmaceutical products, feed, cosmetics, and the like can be suitably added, depending on use.

When the composition for inhibiting myofibrosis of the present invention is provided as a food or beverage, food- or beverage-acceptable components (e.g., food or beverage materials and additives used as needed) can be added to quercetin or a glycoside thereof to provide various foods or beverages (food or beverage compositions). The food or beverage is not limited. Examples include general foods, general beverages, health foods, foods with function claims, foods for specified health uses, foods for the sick, food additives, and ingredients thereof. The form of the food or beverage is also not limited. Examples include various dosage forms of oral solid formulations such as tablets, coated tablets, fine granules, granules, powders, pills, capsules, dry syrups, and chewable tablets, and oral liquid formulations such as oral solutions and syrups. In one embodiment of the present invention, the food or beverage may contain one or more of the followings: the physiologically active ingredient described above such as vitamins, minerals, and nutrients; the proteins and peptides thereof described above; and the amino acids including branched chain amino acids and metabolites thereof.

When the composition for inhibiting myofibrosis of the present invention is provided as a pharmaceutical product or quasi-pharmaceutical product, a pharmacologically acceptable excipient and the like can be added to quercetin or a glycoside thereof to provide various dosage forms of pharmaceutical products (pharmaceutical compositions) or quasi-pharmaceutical products (quasi-pharmaceutical compositions). The form of administration of the pharmaceutical product or quasi-pharmaceutical product is not limited. Oral administration or parenteral administration may be accepted. Oral administration is preferred. The dosage form of the pharmaceutical product or quasi-pharmaceutical product may be one suitable to the form of administration. Examples of the dosage form of the oral pharmaceutical product include oral solid formulations such as tablets, coated tablets, fine granules, granules, powders, pills, capsules, dry syrups, and chewable tablets, and oral liquid formulations such as oral solutions and syrups. Examples of the dosage form of the parenteral pharmaceutical product include injections, infusions, external preparations, suppositories, and percutaneous absorption agents. The pharmaceutical product may be one for non-human animals.

When the composition for inhibiting myofibrosis of the present invention is provided as feed, feed-acceptable components can be added to quercetin or a glycoside thereof to provide feed (feed composition). Examples of the feed include livestock feed for animals such as cows, pigs, chickens, sheep, and horses; feed for small animals such as rabbits, rats, and mice; and pet foods for animal such as dogs, cats, and birds.

When the composition for inhibiting myofibrosis of the present invention is provided as a cosmetic, cosmetically acceptable components such as additives can be added to quercetin or a glycoside thereof to provide a cosmetic (cosmetic composition).

When the composition for inhibiting myofibrosis of the present invention is provided as a food, beverage, pharmaceutical product, quasi-pharmaceutical product, feed, or cosmetic, any production method may be used. For example, the composition can be produced by a usual method using quercetin or a glycoside thereof as an active ingredient.

The composition for inhibiting myofibrosis of the present invention may be labeled with one or more of the following information on a package, container, or package insert: usage, types of active ingredients, the above-described effects, and instructions for use (e.g., ingestion method or administration method). The composition for inhibiting myofibrosis of the present invention may be labeled as having an effect based on the effect of inhibiting the differentiation process from muscle satellite cells into myofibroblasts or the effect of inhibiting myofibrosis. For example, the composition may be labeled as having one or more of the following effects: inhibition, prevention, or amelioration of myofibrosis; improvement of muscle quality; improvement of motor function; increase in muscle mass; and inhibition of muscle atrophy, or may be labeled for use in achieving one or more of the above effects.

The amount of quercetin or a glycoside thereof in the composition for inhibiting myofibrosis of the present invention is not limited, and can be suitably set according to the form and the like. For example, when the composition for inhibiting myofibrosis is provided as a food, beverage, pharmaceutical product, quasi-pharmaceutical product, feed, or cosmetic, regardless of the form, the total amount of quercetin or a glycoside thereof (sum amount of quercetin and a glycoside thereof) in terms of quercetin in the composition is preferably 0.0001% by weight or more, more preferably 0.01% by weight or more, still more preferably 0.1% by weight or more while it is preferably 99.9% by weight or less, more preferably 95% by weight or less, still more preferably 80% by weight or less, particularly preferably 45% by weight or less. In one embodiment, the total amount of quercetin or a glycoside thereof in terms of quercetin in the composition is preferably 0.0001 to 99.9% by weight, more preferably 0.001 to 95% by weight, still more preferably 0.01 to 80% by weight, particularly preferably 0.01 to 45% by weight. In one embodiment, when the composition for inhibiting myofibrosis is provided as a food or beverage, the total amount of quercetin or a glycoside thereof in the food or beverage is preferably 0.0001 to 99.9% by weight, more preferably 0.001 to 45% by weight.

The amount of quercetin or a glycoside thereof can be measured by a known method. For example, a method such as HPLC can be used.

The composition for inhibiting myofibrosis of the present invention can be fed or administered in an appropriate manner according to the form of the composition. The composition for inhibiting myofibrosis of the present invention may be orally administered or fed. Alternatively, the composition may be parenterally administered in the form of an injection or the like. Yet, the composition is preferably orally administered or fed.

The intake (or the amount of administration) of the composition for inhibiting myofibrosis of the present invention is not limited, and may be suitably set according to the form of administration and administration method, for example. In one embodiment, for example, when the composition for inhibiting myofibrosis is orally administered or fed to a human (adult) in order to achieve the effect of inhibiting the differentiation process from muscle satellite cells into myofibroblasts or the effect of inhibiting myofibrosis, the intake of the composition for inhibiting myofibrosis as the total daily intake of quercetin or a glycoside thereof (total daily intake of quercetin and a glycoside thereof) in terms of quercetin is preferably 0.1 mg to 8000 mg, more preferably 0.3 mg to 4000 mg, still more preferably 1.0 mg to 1000 mg, yet still more preferably 10 mg to 500 mg, particularly preferably 10 mg to 200 mg. The above amount is preferably orally administered or fed in one to three portions per day, for example. When the composition for inhibiting myofibrosis is parenterally administered to a human (adult) by injection or the like, the total daily amount of administration of quercetin or a glycoside thereof in terms of quercetin is preferably 0.1 to 8000 mg, more preferably 0.3 mg to 4000 mg, still more preferably 1.0 mg to 1000 mg, yet still more preferably 10 mg to 500 mg, particularly preferably 10 mg to 200 mg.

Preferably, the composition for inhibiting myofibrosis of the present invention is fed or administered to a subject such that the total intake of quercetin or a glycoside thereof is within the range.

In one embodiment, the composition for inhibiting myofibrosis of the present invention preferably contains quercetin or a glycoside thereof in an amount that produces the desired effect of the present invention (i.e., effective amount), in view of form of administration, administration method, and the like. In one embodiment, for example, when the composition for inhibiting myofibrosis is an oral composition such as a food or beverage or an oral pharmaceutical product, the total amount of quercetin or a glycoside thereof in terms of quercetin in the daily intake of the composition per adult is preferably 0.1 to 8000 mg, more preferably 0.3 to 4000 mg, still more preferably 1.0 to 1000 mg, yet still more preferably 10 mg to 500 mg, particularly preferably 10 mg to 200 mg.

A subject to which the composition for inhibiting myofibrosis of the present invention (hereinafter also simply referred to as a “subject”) is administered or fed is preferably an animal, more preferably a mammal (a human or non-human mammal), still more preferably a human. Examples of the non-human mammal include cows, horses, goats, dogs, cats, rabbits, mice, rats, guinea pigs, and monkeys. The subject of administration of the present invention is preferably a subject needing or wanting one or more of the following effects: inhibition of myofibrosis, improvement of muscle quality, improvement of motor function, increase in muscle mass, and inhibition of muscle atrophy. For example, the subject is suitably one with reduced muscle strength due to aging or the like, one wanting to prevent an age-related reduction in muscle strength, or the like.

The present invention also encompasses a method of inhibiting myofibrosis, the method including administering or feeding quercetin or a glycoside thereof. The method of inhibiting myofibrosis is preferably a method of inhibiting myofibrosis by inhibiting the differentiation process from muscle satellite cells into myofibroblasts.

The present invention also encompasses a method of inhibiting the differentiation process from muscle satellite cells into myofibroblasts, the method including administering or feeding quercetin or a glycoside thereof. The methods may be therapeutic methods or non-therapeutic methods. The “non-therapeutic” is a concept that does not include medical activities, i.e., a concept that does not include methods of surgery, therapy, or diagnosis of humans.

The amount of administration of quercetin or a glycoside thereof is not limited as long as it is an amount that produces the effect of inhibiting the differentiation process from muscle satellite cells into myofibroblasts or the effect of inhibiting myofibrosis (i.e., effective amount). Administration or ingestion of the above-described amount, for example, is preferred. Quercetin or a glycoside thereof may be administered or fed directly, or may be administered or fed as a composition containing quercetin or a glycoside thereof. For example, the above-described composition for inhibiting myofibrosis of the present invention can be administered or fed. Quercetin or a glycoside thereof, subject, administration method, amount of administration, preferred embodiments thereof, and the like are the same as those described for the composition for inhibiting myofibrosis. According to the present invention, it is possible to safely inhibit myofibrosis without causing side effects.

The present invention also encompasses the following uses and the like.

Use of quercetin or a glycoside thereof for inhibiting myofibrosis

The use is preferably use of quercetin or a glycoside thereof for inhibiting myofibrosis by inhibiting the differentiation process from muscle satellite cells into myofibroblasts.

Quercetin or a glycoside thereof for use in inhibiting myofibrosis

Use of quercetin or a glycoside thereof for inhibiting the differentiation process from muscle satellite cells into myofibroblasts

Quercetin or a glycoside thereof for use in inhibiting the differentiation process from muscle satellite cells into myofibroblasts

The uses are uses in humans or non-human animals.

The use may be therapeutic or non-therapeutic use.

Preferred embodiments and the like of quercetin or a glycoside thereof and the like are as described above.

In one embodiment, the present invention also encompasses use of quercetin or a glycoside thereof for producing a composition for inhibiting myofibrosis. Preferred embodiments of the composition for inhibiting myofibrosis are the same as described above. The present invention also encompasses use of quercetin or a glycoside thereof for producing a composition for inhibiting the differentiation process from muscle satellite cells into myofibroblasts.

### EXAMPLES

The present invention is described in more detail below with reference to examples which do not intend to limit the scope of the present invention.

### Example 1

Differentiation Induction of Rat Skeletal Muscle-Derived Muscle Satellite Cells into Myofibroblasts

### (1) Isolation of Muscle Satellite Cells and Culturing Conditions

Gastrocnemius, soleus, plantaris muscle, tibialis anterior, extensor digitorum longus, and quadriceps were extracted from male Fischer 344 rats of 9 to 15 weeks old under anesthesia, and minced in phosphate buffered saline (PBS) (Life Technologies Corporation) on ice. The minced muscle tissue was enzymatically degraded by Protease (Sigma-Aldrich) at 37° C., and then centrifuged repeatedly, whereby the muscle satellite cells were isolated. The muscle satellite cells were suspended in Dulbecco's Modified Eagle Medium (DMEM) containing 10% fetal equine serum, and cultured for 24 hours. After culturing, centrifugation was performed again, and the deposited muscle satellite cells were seeded onto an 8-well slide glass (Thermo Fisher Scientific) to a density of 5×10⁴ to 7×10⁴ cells/mL in F-10 medium (Gibco) containing 20% bovine serum. After seeding, the muscle satellite cells were cultured for 72 hours or 120 hours. The muscle satellite cells for tissue staining were cultured for 72 hours, and muscle satellite cells for genetic analysis were cultured for 120 hours to provide enough cells. In the case of the muscle satellite cells to be cultured for 120 hours, the F-10 medium containing 20% bovine serum was replaced 72 hours after seeding.

(2) Differentiation (Fibrosis)-Inducing Conditions of Muscle Satellite Cells into Myofibroblasts

After the culturing for 72 hours or 120 hours, the muscle satellite cells were cultured in a differentiation-inducing medium for 24 hours. The differentiation-inducing medium was a medium in which a TGF β (TGF-β) signal inhibitor, quercetin, or solvent was added in an amount shown in Table 1 to Dulbecco's Modified Eagle Medium (DMEM) containing 2% fetal equine serum. TGF β (Sigma-Aldrich) was used for fibrosis induction. The compound was added to the differentiation-inducing medium using HCl as solvent to a final concentration of 1 ng/mL. TGF β was added when a TGF β signal inhibitor or quercetin was added. The TGF β signal inhibitor was SB525334 (6-[2-tert-butyl-5-(6-methyl-pyridin-2-yl)-1H-imidazol-4-yl]-quinoxaline available from Wako Pure Chemical Industries, Ltd.). SB525334 was added using dimethyl sulfoxide (DMSO) as solvent to a final concentration of 1 μM. DMSO was also used as solvent when quercetin was added.

TABLE 1

| Group | TGF β | HCl | DMSO | SB525334 | Quercetin |
|---|---|---|---|---|---|
| TGFβ(−) | — | 4 μM | 0.1% (v/v) | — | — |
| TGFβ(+) | 1 ng/mL | — | 0.1% (v/v) | — | — |
| TGFβ(+) + | 1 ng/mL | — | — | 1 μM | — |
| SB525334 | | | | | |
| TGFβ(+) + | 1 ng/mL | — | — | — | 50 μM |
| quercetin 50 μM | | | | | |
| TGFβ(+) + | 1 ng/mL | — | — | — | 100 μM |
| quercetin 100 μM | | | | | |

### (3) Evaluation of Morphological Changes by Fibrosis Induction

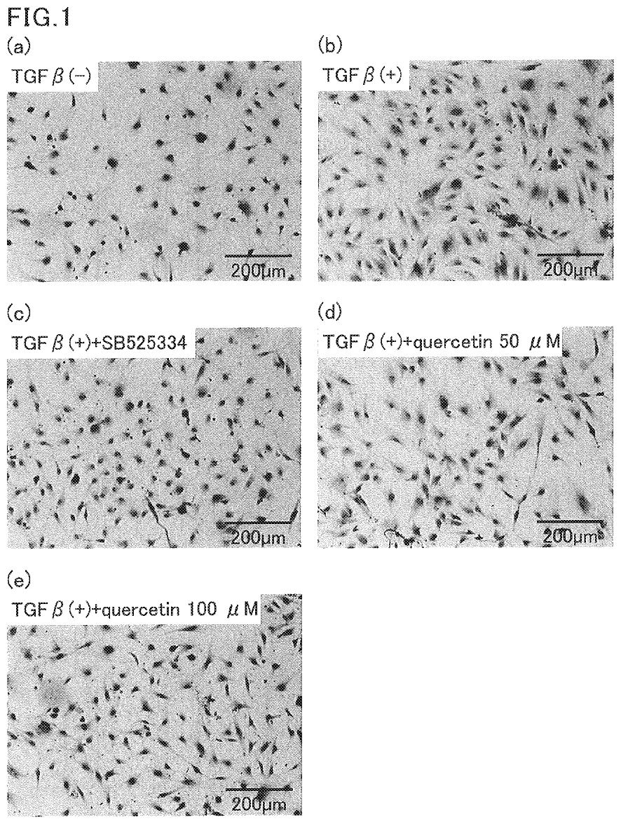

The cells cultured in the differentiation-inducing medium were washed with PBS, and then fixed in 10% formalin (Wako Pure Chemical Industries, Ltd.) at room temperature for 10 minutes. Subsequently, the cells were reacted with Bouin's solution (Sigma-Aldrich) at 56° C. for 15 minutes, and stained with Masson's Trichrome using Trichrome Stains (MASSON) Kit (Sigma-Aldrich). FIG. 1 shows micrographs of stained samples.

### (4) Fibrosis Marker Gene Expression Analysis

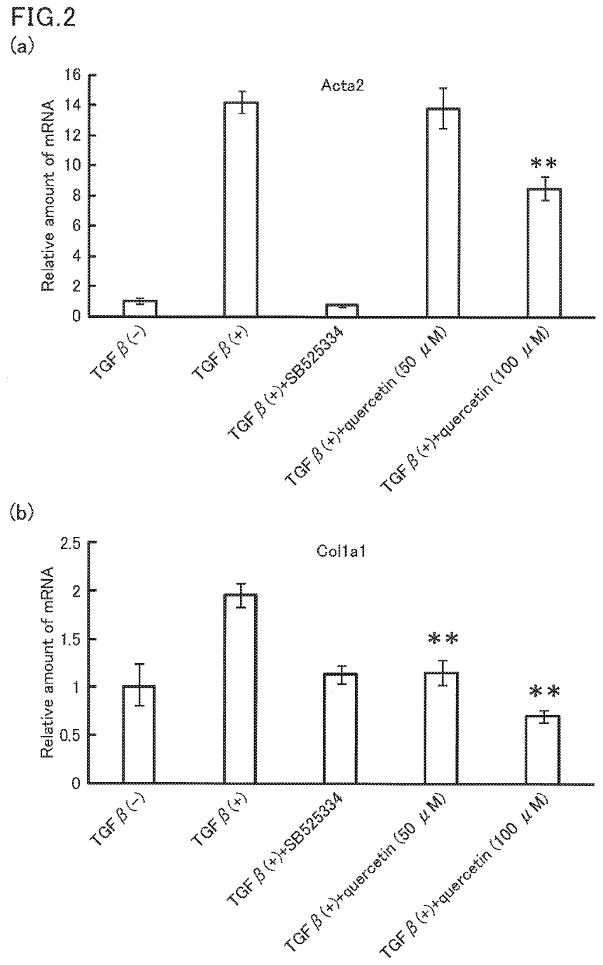

Using an RNeasy micro kit (Qiagen), RNAs were prepared from the cells cultured in the differentiation-inducing medium. The prepared RNAs whose concentration was made uniform was heat-treated at 70° C. for 2 minutes, and then quenched for use. Reverse transcription reaction was performed using 15 ng of extracted RNAs at the following temperatures: 25° C. for 10 minutes, 37° C. for 120 minutes, 85° C. for 5 minutes, and then 4° C. for cooling. The cDNAs obtained by reverse transcription reaction was subjected to quantitative PCR in Step One Plus Real Time PCR System using TaqMan Fast Universal PCR Mastermix (Life Technologies Corporation). The expression levels of Colla1 gene and Acta2 gene as a myofibroblast marker were measured. For quantitative PCR, the cells were first maintained at 95° C. for 20 seconds, and then subjected to 40 cycles of reaction at 95° C. for 1 second and 60° C. for 20 seconds. The expression level of 18S rRNA gene as an internal standard gene of each group was measured, and relative values of Colla1 gene expression and Acta2 gene expression were calculated from the Ct value (the number of cycles required until cells were amplified to a certain amount) of each of 18S rRNA gene (Applied Biosystems: Hs99999901_s1), Colla1 gene (Applied Biosystems: Rn01463848_m1), and Acta2 gene (Applied Biosystems: Rn01759928_g1). FIG. 2 shows the results.

FIG. 1 shows micrographs of muscle satellite cells stained with Masson's Trichrome after being cultured for 24 hours in a differentiation-inducing medium with addition of a TGF β signal inhibitor or quercetin. In FIGS. 1(a) to 1(e), FIG. 1(a) is a TGF β-free group (TGF β(−)), FIG. 1(b) a TGF β-added group (TGF β(+)), FIG. 1(c) is a SB525334-added group (TGF β(+)+SB525334), FIG. 1(d) is a quercetin 50 μM-added group (TGF β(+)+quercetin 50 μM), and FIG. 1(e) is a quercetin 100 μM-added group (TGF β(+)+quercetin 100 μM). The scale bar in each of FIGS. 1(a) to 1(e) represents 200 μm. In the TGF β-free group (FIG. 1(a)), the cells show a spherical-like morphology (cytoplasm is not stretched). In contrast, in the TGF β-added group (FIG. 1(b)), the cells clearly changed morphology to myofibroblasts (cytoplasm is stretched). Addition of SB525334 that is a TGF β signal inhibitor clearly inhibited TGF β-induced morphological changes of muscle satellite cells to myofibroblasts (FIG. 1(c)). In the 50-μM quercetin-added group, morphological changes of muscle satellite cells to myofibroblast-like cells was more inhibited (FIG. 1(d)) than in the TGF β-added group (FIG. 1(b)). The 100-μM quercetin-added group showed a stronger effect of inhibiting morphological changes of muscle satellite cells to myofibroblasts (FIG. 1(e)), and this effect was stronger than the 50-μM quercetin-added group (FIG. 1(d)). Thus, the results qualitatively show that quercetin has an effect of inhibiting morphological changes of muscle satellite cells to myofibroblasts under TGF-β-mediated fibrosis-inducing conditions.

FIG. 2 shows analysis results of the expression level of an intracellular fibrosis marker gene after muscle satellite cells were cultured for 24 hours in a differentiation-inducing medium with addition of a TGF β signal inhibitor or quercetin ((a): Acta2 gene; (b) Colla1 gene). Significance of the results shown in FIGS. 2(a) and 2(b) was tested by Dunnett's test (**: P<0.01, vs. TGF β(+)). The relative amounts of mRNA shown in FIGS. 2(a) and 2(b) are the amounts of mRNA of the genes, respectively, relative to the amounts of mRNA of these respective genes in the TGF β-free group (TGF β(−)) taken as 1.

The expression of Acta2 gene that is expressed specifically in myofibroblasts was found to be significantly inhibited in the SB525334-added group. The expression of Acta2 gene was also significantly inhibited in the 100-μM quercetin-added group. The expression of Colla1 gene was found to be significantly inhibited in the SB525334-added group, and was also found to be inhibited in a concentration-dependent manner in the quercetin-added group. Thus, the results quantitatively show that quercetin inhibits the differentiation process from muscle satellite cells into myofibroblasts.

The following shows production examples of the composition for inhibiting myofibrosis of the present invention.

### (Production Example 1) Tablets

Quercetin glucoside 10 g

### Vitamin E 50 g

### Starch 222 g

Sucrose fatty acid ester 9 g
Silicon oxide 9 g

These components were mixed together and tableted using a single-shot tableting machine, whereby tablets each having a diameter of 9 mm and a mass of 300 mg were produced

### (Production Example 2) Health Drink

### Sodium DL-tartrate 0.1 g

Succinic acid 0.009 g
Liquid sugar 800 q
Citric acid 12 g

### Vitamin C 10 g

Quercetin glucoside 1 g

### Vitamin E 20 g

### Cyclodextrin 5 g

### Emulsifier 5 g

### Perfume 15 g

Potassium chloride 1 g
Magnesium sulfate 0.5 g

The above components were mixed together, and water was added to adjust the total volume to one liter. The thus-obtained health drink is to be consumed in an amount of 100 mL or more per time.

### INDUSTRIAL APPLICABILITY

The composition for inhibiting myofibrosis of the present invention contains quercetin or a glycoside thereof. Thus, the composition can inhibit the differentiation process from muscle satellite cells into myofibroblasts and can inhibit myofibrosis. The composition for inhibiting myofibrosis of the present invention can also inhibit muscle atrophy or the like, owing to the effect of quercetin or a glycoside thereof to inhibit the differentiation process from muscle satellite cells to myofibroblasts. Quercetin or a glycoside thereof is contained in ingestible plants, and no side effects in healthy adults have been reported so that the safety is ensured. Thus, the composition for inhibiting myofibrosis of the present invention is considered to be safely and continuously consumable and capable of inhibiting a reduction in muscle quality, a reduction in muscle mass, and muscle atrophy which are induced by myofibrosis and contributing to improvement in motor function. Thus, the composition is likely to be highly industrially applicable.

1. A composition for inhibiting myofibrosis, comprising:
quercetin or a glycoside thereof as an active ingredient.

2. The composition for inhibiting myofibrosis according to claim 1, wherein the composition inhibits the differentiation process from muscle satellite cells into myofibroblasts.

3. The composition for inhibiting myofibrosis according to claim 1, wherein the composition has an effect of improving muscle quality.

4. The composition for inhibiting myofibrosis according to claim 1, wherein the composition has an effect of improving motor function.

5. The composition for inhibiting myofibrosis according to claim 1, wherein the composition has an effect of increasing muscle mass.

6. The composition for inhibiting myofibrosis according to claim 1, wherein the composition has an effect of inhibiting muscle atrophy.

7. The composition for inhibiting myofibrosis according to claim 1, wherein the composition has an effect of inhibiting accumulation of extracellular matrix in muscle.

8. The composition for inhibiting myofibrosis according to claim 7, wherein the extracellular matrix is at least one of collagen or an advanced glycation end product.

9. The composition for inhibiting myofibrosis according to claim 1, which is a food, beverage, or oral pharmaceutical product.

10. The composition for inhibiting myofibrosis according to claim 1, wherein the composition is labeled as having one or more of the following effects:
inhibition, prevention, or amelioration of myofibrosis;
improvement of muscle quality;
improvement of motor function;
increase in muscle mass; and
inhibition of muscle atrophy, or
is labeled for use in achieving one of more the above effects.

11-12. (canceled)

13. A method of inhibiting myofibrosis, comprising:
administering or feeding quercetin or a glycoside thereof.

14. A method of inhibiting the differentiation process from muscle satellite cells into myofibroblasts, comprising:
administering or feeding quercetin or a glycoside thereof.
